(19) United States
(12) Patent Application Publication
(10) Pub. No.: US 2012/0191156 A1
(43) Pub. Date: Jul. 26, 2012

(54) Treatment of various ailments

(76) Inventor: Donald A. Rhodes, (US)

(21) Appl. No.: 13/506,116

(22) Filed: Mar. 27, 2012

Related U.S. Application Data
(63) Continuation of application No. 12/807,798, filed on Sep. 13, 2010, now abandoned.
Application No. 12/807,798 is a division of application No. 11/326,230, filed on Jan. 5, 2006, now abandoned.

(51) Int. Cl. A61N 1/36

(52) U.S. Cl. 607/17; 607/62; 607/52; 607/42; 607/45; 607/46; 607/39; 607/40; 607/50; 607/48

(57) ABSTRACT

A method of treating a wide variety of heretofore considered unrelated ailments comprises delivering electricity through a circuit in the body. The circuit includes at least four nerves leading from at least two of the patient's extremities to various nerve roots adjacent the spinal column. Electrical energy from an electrical interferential therapy device is delivered through electrodes on the extremities adjacent the nerve endings until symptoms of the diagnosed ailment ameliorates. Sending impulses from the periphery to the central nervous system appears to the help the body manufacture various neuropeptides and other chemicals which control the essential basics of the body's health and well being.

This application is a continuation of application Ser. No. 12/807,798, filed Sep. 13, 2010, which is a division of application Ser. No. 11/326,230 filed Jan. 5, 2006.

This invention comprises a method of treating the human body for various ailments, many of which have heretofore been thought to be unrelated.

### BACKGROUND OF THE INVENTION

This invention is an improvement and extension of the teachings of U.S. Pat. No. 5,995,873.

As disclosed in U.S. Pat. No. 5,995,873, it is known in the art to treat pain or other symptoms by the application of energy in the form of alternating current electricity, magnetism and other forms of electromagnetic energy. One well accepted technique is known as electrical interferential therapy in which electrodes are connected in a crossing pattern adjacent the area to be treated. Alternating current is delivered through the electrodes into the body. Such devices are commercially available from Rehabilicare Corporation of St. Paul, Minn. and Dynatronics, Inc. of Salt Lake City, Utah. This type equipment is used to treat small areas of the body because the electrodes are spaced relatively close together in the region to be treated.

### SUMMARY OF THE INVENTION

It has been discovered that the application of electrical interference therapy essentially as shown in U.S. Pat. No. 5,995,873 is effective in treating diseases or ailments of a rather wide variety which have previously been thought to be unrelated.

In this invention, patients are diagnosed with an ailment and then treated in accordance with this invention. Typically, treatments are repeated periodically until symptoms of the ailment diminish or ameliorate. Ailments treatable by this invention fall into several categories: (1) situations where the predominate mechanism is abnormal carbohydrate metabolism; (2) situations where the predominate mechanism is poor to mediocre circulation; (3) situations where the predominate mechanism is abnormal inflammation and (4) situations where the predominate mechanism is a poorly functioning immune system. After the ailment is diagnosed, a treatment is designed or selected. Broadly, electrodes are attached to the patient's extremities adjacent nerve endings and low frequency electrical current is passed through the electrodes.

It is accordingly an object of this invention to provide an improved technique for treating patients for a variety of ailments.

Another object of this invention is to provide an improved method for treating patients by delivering electromagnetic energy through nerves leading from the spinal column to the extremities.

These and other objects and advantages of this description will become more apparent as this description proceeds, reference being made to the accompanying drawings and appended claims.

### BRIEF DESCRIPTION OF THE DRAWINGS

FIG. 1 is a pictorial view of a patient showing, for clarity of illustration, part of the circuit established by practice of a first embodiment of this invention;

FIG. 2 is a pictorial view, similar to FIG. 1, showing the remainder of the circuit established by the first embodiment of this invention;

FIG. 3 is a pictorial view, similar to FIGS. 1 and 2, showing another circuit established by practice of a second embodiment of this invention;

FIG. 4 is a pictorial view showing another circuit established by practice of another embodiment of this invention;

FIG. 5 is a pictorial view showing another circuit established by practice of another embodiment of this invention; and

FIG. 6 is a schematic view of a conventional electrical interferential therapy device.

### DETAILED DESCRIPTION

This treatment is for diseases with or without chronic pain. In this invention, patients are diagnosed with an ailment and then treated in accordance with this invention. Treatments are repeated periodically until symptoms of the ailment diminish or ameliorate. The treatment of this invention is designed to normalize various bodily functions including normalizing abnormal circulation, normalizing abnormal carbohydrate metabolism, decreasing inflammation and/or normalizing decreased immune system function.

Without being bound by any particular theory, the current working hypothesis is that the treatment of this invention is effective due to a combination of the following aspects of the treatment: low frequency electrical current passing through long sections of nerves; electrode pad placement; production of cyclic adenosine monophosphate; the choice of the peripheral nerves being stimulated so there is a cross-over effect in the central nervous system; leakage of action potentials from the nerves being stimulated into nerves entering the sympathetic ganglia; the quadrilateral location of stimulation; creation of action potentials in peripheral nerves being stimulated; activation of the sodium pump in the nerves being stimulated; production of ACTH; production of dynorphins, enkephalins or beta-endorphins; creation of action potentials in sympathetic fibers within the peripheral nerves being stimulated, which enter the sympathetic ganglia directly; analgesia causing a reduction in the production of substance P; production of melatonin; and/or the production of circulation altering neuropeptides such as vasoactive intestinal polypeptide (VIP) and calcitonin gene-related peptide (CGRP).

By applying electrical currents in accordance with this invention, the treatment of this invention is effective for chronic pain but also many diseases and ailments which may or may not be painful and which heretofore have been thought to be unrelated.

Ailments treatable by this invention fall into several categories: (1) situations where the predominate mechanism is abnormal carbohydrate metabolism; (2) situations where the predominate mechanism is poor to mediocre circulation; (3) situations where the predominate mechanism is abnormal inflammation and (4) situations where the predominate mechanism is a poorly functioning immune system. While most diseases and ailments treatable by this invention involve all or most of these abnormalities, most diseases are primarily caused by one or two of these abnormalities. This is not to say that the ultimate cause of the ailments treatable by this invention are those abnormalities listed above. Typically, the ultimate cause is some other factor, often genetics, that creates the abnormalities which this invention can ameliorate.

Abnormal carbohydrate metabolism is believed responsible, or at least substantially responsible, for Alzheimer's disease, cancer, diabetes type 1 and type 2, narcotic withdrawal, fibromyalgia, Gulf War Syndrome, hypoglycemia, insomnia, irregular heart beat, Irritable Bowel Syndrome, depression, bipolar disorder, low testosterone levels, panic attacks, Parkinson's disease, sleep apnea, and skin ulcerations.

Abnormal circulation is believed responsible, or at least substantially responsible, for Brittle Bone Disease, Crohn's disease, cystic fibrosis, diabetes type 1 and type 2, diabetic retinopathy, endometriosis, erectile dysfunction or impotence, fibromyalgia, gastroesophageal reflux disease commonly known as GERD's, glaucoma, Gulf War Syndrome, hypercholesterolemia, hypertension, hypertriglyceridemia, hypoglycemia, insomnia, irregular heart beat, Irritable Bowel Syndrome, depression, bipolar disorder, low testosterone levels, osteoporosis, panic attacks, Parkinson's disease, peripheral vascular disease, psoriasis, sickle cell anemia, sleep apnea, skin ulcerations, TMJ Disease, trigeminal neuralgia and ulcerative colitis.

Abnormal inflammation is believed responsible, or at least substantially responsible, for AIDS, ALS, Alzheimer's disease, cancer, congestive heart failure, Gulf War Syndrome, hypercholesterolemia, hypertension, hypertriglyceridemia, irregular heart beat, Irritable Bowel Syndrome, Parkinson's disease, psoriasis, sickle cell anemia, skin ulcerations, TMJ Disease and ulcerative colitis.

Abnormal immune function is believed, responsible, or at least substantially responsible, for AIDS, cancer, Crohn's disease, cystic fibrosis, diabetes type 1 and type 2, endometriosis, fibromyalgia, Gulf War Syndrome, Irritable Bowel Syndrome, skin ulcerations and ulcerative colitis.

In this invention, an electric circuit is established in the patient's body. The circuit includes at least two, often four and ideally eight segments. Each segment includes the nerve fibers having one terminus on an extremity, i.e. on the foot or hand of the patient, and one terminus adjacent a sympathetic nerve ganglia near a nerve ganglia adjacent a particular vertebra on the spinal column. Some type of electrical connection, the exact details of which are as yet unknown, is made between the nerve ganglia adjacent the spinal column. It is clear that the circuit includes the nerve fibers which extend from the more distal aspect of a first peripheral nerve to its root adjacent the spinal column. It is believed the circuit continues through the root of the first nerve into the spinal column, through the spinal column and exits from the spinal column through the root of a second nerve. The circuit continues through the fibers of the second peripheral nerve to a more distal aspect of that nerve.

The energy delivered through the circuit may be selected from a variety of electromagnetic types. Although interferential electrical energy has so far been shown to be the most desirable, H-wave stimulation with a physical therapy device such as made by Electronic Waveform Lab, Huntington Beach, Calif., galvanic stimulation with a physical therapy device such as a Model SW made by Rehabilicare Corporation, St. Paul, Minn., SMP stimulation with a physical therapy device which creates constantly changing TENS frequency such as made by Rehabilicare Corporation, St. Paul, Minn., or matrix electrostimulation with a physical therapy device such as made by Rehabilicare Corporation, St. Paul, Minn., have shown desirable effects.

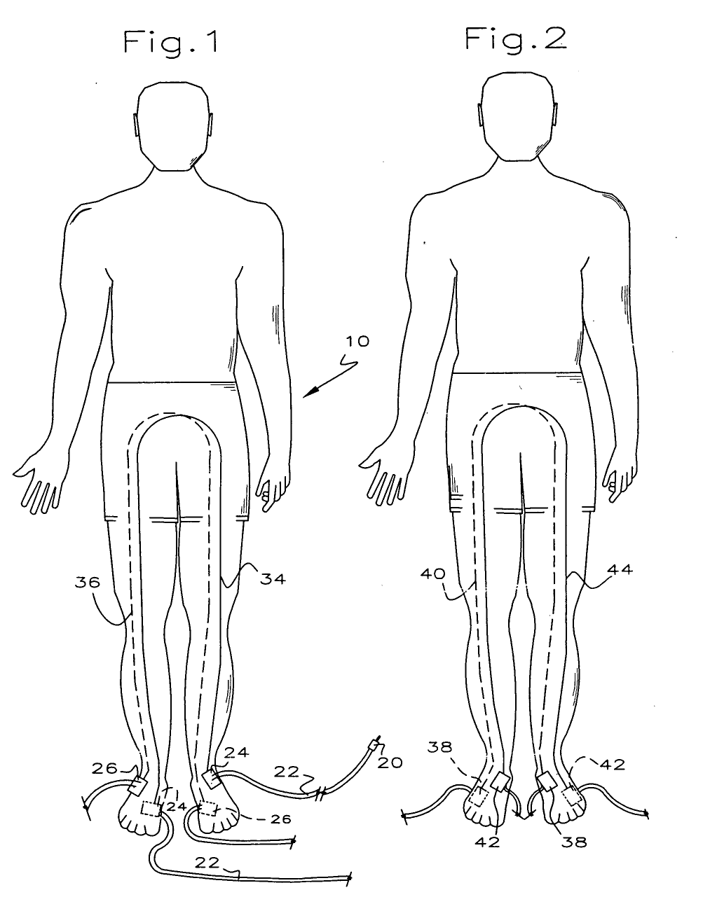

Referring to FIGS. 1 and 6, a patient 10 is illustrated as being treated in accordance with this invention by a conventional electrical interferential therapy device 12 such as available commercially from Rehabilicare Corporation of St. Paul, Minn. The device 12 includes a control panel 14 with output jacks 16, 18 capable of accepting a jack 20 of an insulated wire pair 22 leading to electrode pairs 24, 24′ and 26, 26′. The dial 28 of the device 12 controls the amperage delivered to the electrodes and is set to deliver maximum amperage consistent with patient comfort. The electrode switch 30 is set to either two or four depending on whether one or two pair of electrodes are being used. The setting of the frequency switch 32 is subject to some adjustment. The frequency switch 32 controls the “beat” frequency. For example, if the setting is at ten, the patient receives ten beats per second. In fact, the frequency of the alternating current delivered by the device of Rehabilicare is nominally 4000 Hz and the frequency switch 32 acts to vary the frequency, at a setting of ten, to 4010 Hz. In the event a more complete understanding of the device 12 is necessary, reference is made to appropriate publications of Rehabilicare Corporation. In this invention, the settings of the frequency switch 32 is normally below ten, and preferably below five and is optimally at four.

The electrodes 24, 26 are attached to the patient's skin in a conventional manner, i.e. they are self adherent. The location of the electrodes 24, 26 on the patient establish the electrical circuit in the patient's body. As shown in FIG. 1, in one technique, one electrode 24 is placed adjacent the end or terminus of the right medial plantar nerve L5 and its matching electrode or mate 24′ is placed adjacent the end or terminus of the left sural nerve S1, inferior to the left ankle bone (lateral malleolus) thereby establishing or creating a first circuit 34 in the patient's body. As used herein, the reference characters L5, S1 and the like are standard medical terminology for the nerve. Those skilled in the art will recognize L5 as being the nerve which extends away from the fifth lumbar vertebra and S1 as being the nerve which extends away from the first sacral vertebra.

Those skilled in the art will recognize that the terminus of the right medial plantar nerve L5 is located on the bottom of the right foot, approximately on the ball of the foot. The terminus of the left sural nerve S1 is located below the left ankle bone (lateral malleolus). Another electrode 26 is placed adjacent the terminus of the right sural nerve S1 and its matching electrode or mate 26′ is placed adjacent the terminus of the left medial plantar nerve L5 thereby establishing a second circuit 36 in the patient's body. Turning the device 12 on delivers electrical energy through the circuits 34, 36. Experience has shown a decrease in pain in patients complaining of pain and a decrease in symptoms consistent with an imbalanced sympathetic nervous system. Those skilled in the art will recognize that the medial plantar nerves L5 and the sural nerves S1 terminate adjacent the spinal column near adjacent spinal vertebra, in the area of the connection to the lumbar sympathetic ganglia.

Preferably, a second electrical interferential therapy device 12′ is used simultaneously with the first device 12 and another set of circuits is simultaneously established as suggested in FIG. 2. The electrical interferential therapy device 12′ is either a separate unit from the device 12 or they may be incorporated together in a single housing. In any event, an electrode 38 is attached to the patient's skin adjacent the terminus of the right lateral plantar nerve L5 and its matching electrode or mate 38′ is placed adjacent the terminus of the left saphenous nerve L4, at the ankle, thereby establishing a circuit 40. Those skilled in the art will recognize that the terminus of the right lateral plantar nerve L5 is on the bottom of the right foot below the little toe and the fourth toe on the pad of the foot near the fifth metatarsal head. The terminus of the left saphenous nerve L4 is on the top inside (medial-anterior aspect) of the left ankle. An electrode 42 is attached to the patient's skin adjacent the terminus of the right saphenous nerve L4 and its matching electrode or mate 42′ is placed adjacent the terminus of the left lateral plantar nerve L5 thereby establishing a circuit 44. Those skilled in the art will recognize that the terminus of the right saphenous nerve L4 is on the top inside of the right foot, forward of the right ankle. The terminus of the left lateral plantar nerve is on the bottom of the left foot below the little toe and the fourth toe on the pad of the foot. Turning the device 12 on delivers electrical energy through the circuits 40, 44. The techniques of FIGS. 1 and 2 are preferably run simultaneously with similar or identical settings on the devices 12, 12′. Those skilled in the art will recognize that the lateral plantar nerves L5 and the saphenous nerves L4 terminate adjacent the spinal column near adjacent spinal vertebrae.

Another technique is shown in FIG. 3. An electrode 46 is placed adjacent the end or terminus of the nerve L5 of the left forefoot plantar aspect. Its matching electrode or mate 46′ is placed adjacent the end or terminus of the right cranial nerve C8 where the fifth finger joins the right hand thereby establishing or creating a first circuit 48 in the patient's body. Another electrode 50 is placed adjacent the terminus of the left sural nerve S1 on the lateral aspect of the left ankle and its matching electrode or mate 50′ is placed adjacent the terminus of the right cranial nerve C6 where the thumb joins the hand thereby establishing a second circuit 52 in the patient's body. Turning the device 12 on delivers electrical energy through the circuits 48, 52. Those skilled in the art will recognize that the nerves L5 and the sural nerves S1 terminate adjacent the spinal column near adjacent spinal vertebra, in the area of the connection to the lumbar sympathetic ganglia while the nerves C6, C8 terminate adjacent the spinal column near spinal vertebrae that are far above the termini of the nerves L5, S1.

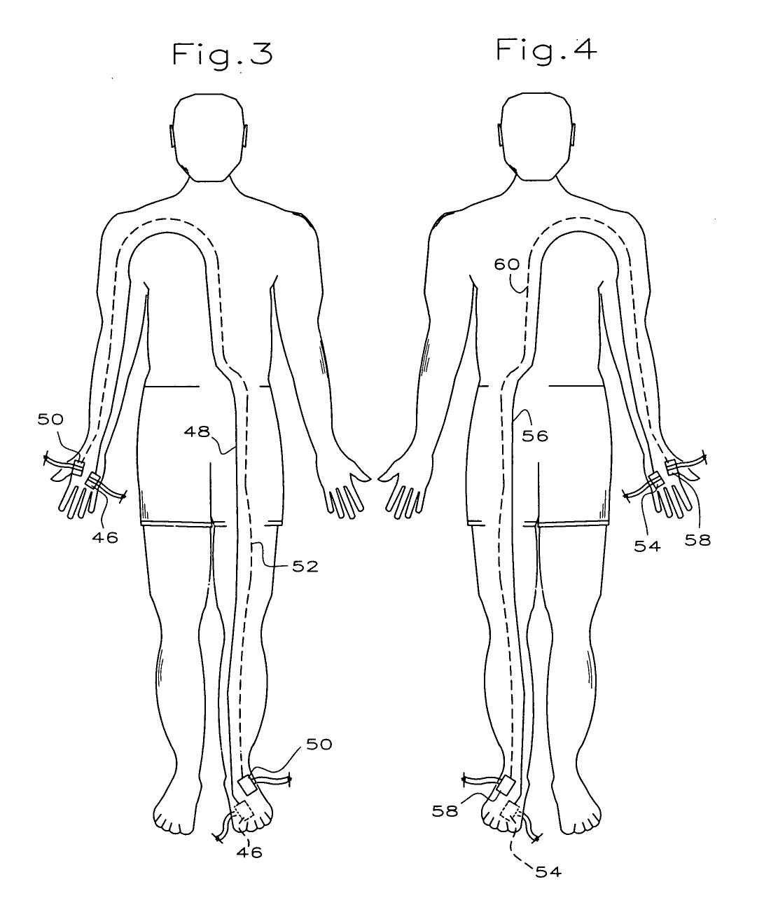

A second electrical interferential therapy device 12′ may be used simultaneously with the first device 12 and another set of circuits may simultaneously established as suggested in FIG. 4 so the circuits of FIGS. 3 and 4 are normally used together. An electrode 54 is attached to the patient's skin adjacent the terminus of the nerve L5 on the right forefoot planar aspect and its matching electrode or mate 54′ is placed adjacent the terminus of the cranial nerve C8 on the left palmer surface where the fifth finger joins the hand thereby establishing a circuit 56 in the patient's body. An electrode 58 is attached to the patient's skin adjacent the terminus of the right sural nerve S1 and its matching electrode or mate 58′ is placed on the left palmer surface adjacent the terminus of the left cranial nerve C6 thereby establishing a circuit 60. Turning the device 12′ on delivers electrical energy through the circuits 56, 60. The techniques of FIGS. 1 and 2 are preferably run simultaneously with similar or identical settings on the devices 12, 12′. Those skilled in the art will recognize that the nerves L5 and the sural nerves S1 terminate adjacent the spinal column near adjacent spinal vertebra, in the area of the connection to the lumbar sympathetic ganglia while the nerves C6, C8 terminate adjacent the spinal column near spinal vertebrae that are far above the termini of the nerves L5, S1.

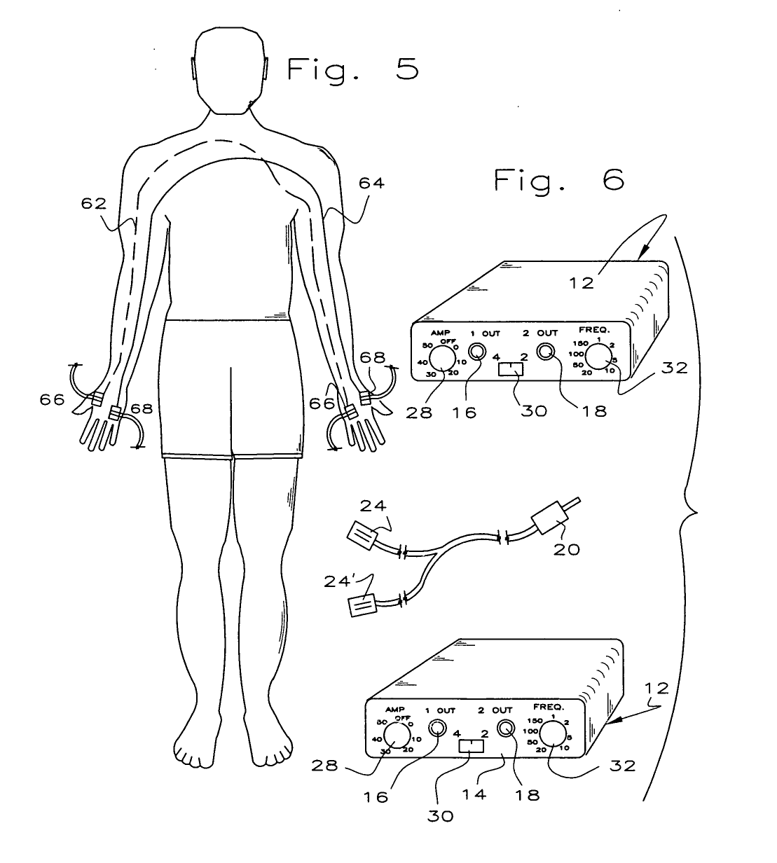

Referring to FIG. 5, another set of circuits 62, 64 is established. An electrode 66 is adhesively placed at the terminus of the cranial nerve C8 on the left palmer surface where the fifth finger joins the hand and its matching electrode 66′ is placed at the terminus of the cranial nerve C6 on the right palmer surface where the thumb joins the hand thereby establishing the circuit 62. An electrode 68 is attached adjacent the terminus of the cranial nerve C6 on the left palmar surface where the thumb joins the hand and its matching electrode or mate 68′ is placed adjacent the terminus of the cranial nerve C8 on the right palmer surface where the fifth finger joins the hand thereby establishing the circuit 64.

### Case Study 1

A 47 year old Caucasian male had been diagnosed as having diabetes type 2 for eight years. The patient developed erectile dysfunction/impotence three years after the diagnosis of diabetes. After 10 days of treatments, this problem disappeared.

### Case Study 2

A 69 year old Caucasian woman was diagnosed as having cataracts. The woman received the treatments twice a day for two months. She had been scheduled for cataract surgery. However, when her opthamologist examined her, she was advised that she no longer had cataracts and, therefore, the surgery was cancelled.

### Case Study 3

A 45 year old Caucasian woman had been advised years previously that she had glaucoma. She had tried a variety of prescription medications, without improvement. Within 30 days of treatment the patient's ophthalmologist retested the patient's intraocular pressure and found that the pressure had normalized.

### Case Study 4

A 24 year old Caucasian woman had been diagnosed as having Diabetes type 1 when she was 14 years old. Following 30 days of treatments, the patient reported that she was using less than one half the amount of insulin that she had used prior to the treatments.

### Case Study 5

A 29 year old veteran of the Gulf War had all of the symptoms of Gulf War Syndrome. However, after 30 days of treatment, the patient no longer had any symptoms of Gulf War Syndrome.

### Case Study 6

A 14 year old Caucasian girl had been diagnosed as having Bipolar Disorder. Numerous medications had been utilized without success. She was given the treatments twice per day and after 14 days of treatment, she no longer exhibited the behaviors which resulted in her diagnosis of Bipolar Disorder.

### Case Study 7

A 60 year old Caucasian man had been diagnosed as having Diabetes type 2 for many years was advised by an ophthalmologist that he had significant diabetic retinopathy. However, after three months of treatments, he was advised that he no longer had the diabetic retinopathy.

### Case Study 8

A 44 year old Caucasian woman was diagnosed as having severe hypercholesterolemia. However, after receiving 90 days of treatments follow-up blood tests showed that her cholesterol level was nearly normal.

### Case Study 9

A 50 year old Caucasian an was diagnosed as having severe hypertriglyceridemia. However, after receiving 90 days of treatments follow-up blood tests showed that his triglyceride level was nearly normal.

### Case Study 10

A 62 year old Caucasian man was diagnosed as having Alzheimer's disease. He received twice a day treatments for 30 days. At the end of that time his daughter said her father was able to read the newspaper and carry on a normal conversation and that he was unable to do either of these before beginning treatments.

### Case Study 11

A sixty five year old Caucasian woman was diagnosed as having ovarian cancer. The patient received once per day treatments for two months. After one month, she was able to discontinue her wheelchair. After two months of treatment, a CT scan was no longer able to identify a malignant lesion.

### Case Study 12

A 48 year old Hispanic man was diagnosed as having sleep apnea, on the basis of sleep studies. The patient began twice per day treatments for one month and, thereafter, once per day treatments for four months. At the end of this time the patient had sleep studies, which showed that he no longer had sleep apnea.

### Case Study 13

A six year old Caucasian boy was diagnosed as having Autism at age 5. He received nightly treatments for 20 minutes through his feet and hands. Normally, his parent would have to drag him up the sidewalk to the special school he attended. However, the day following his first treatments he walked into the Autism school without objection. In addition, within two weeks, he was trying to verbalize much more than he was prior to beginning treatments.

### Case Study 14

A 44 year old Hispanic woman had suffered from insomnia for more than 20 years and only slept fitfully for three to four hours per night. The patient received twice per day treatments for one month. By the end of the month of treatment, the patient was sleeping six to eight hours per night and reported sleeping very soundly.

### Case Study 15

A 66 year old Caucasian man had a two centimeter ulceration, on the dorsum of this right foot. The patient began twice per day treatments for one month. At the end of this time the ulceration was completely healed.

### Case Study 16

A cardiologist diagnosed a 66 year old Caucasian man as having an irregular heart beat and placed upon appropriate medication. In spite of this medication, he continued to exhibit irregular heat beating. The patent began twice per day treatments. Within two weeks, his heart rate became regular. After another month, he was able to stop taking the medication, without resumption of his irregular heartbeat.

### Case Study 17

A 64 year old Caucasian man had been diagnosed as having Diabetes type 2 more that 25 years before. Prior to beginning treatments, his blood sugar was very high, in spite of, taking insulin. After six months of treatments, his blood sugar was low, in spite of, taking no insulin.

### Case Study 18

A 45 year old Hispanic man had a chief complaint of grinding his teeth, while sleeping, which was verified by his wife. The patient began twice per day treatments for one month. At the end of this time the patient's wife stated that he no longer ground his teeth while sleeping.

### Case Study 19

A 38 year old Caucasian woman was diagnosed as having Irritable Bowel Syndrome. The patent suffered from diarrhea and constipation. The patient began twice per day treatments. Within one month, the diarrhea and constipation stopped.

### Case Study 20

A 24 year old Caucasian woman was diagnosed as having TMJ disease. The patient began twice per day treatments for one month and, thereafter, one treatment per day for two months. AT the end of this time the patient had no symptoms of TMJ.

### Case Study 21

A 64 year old Caucasian man was being treated for kidney failure due to hypertension and diabetes. He had been advised that this kidneys were failing and that he only had approximately 50% or normal kidney function. A Nephrologist advised the patient that the patient should expect that he would be on renal dialysis within 2 years. The patient began twice per day treatments for one month followed by once per day treatment thereafter. After nine months of treatments his Nephrologist advised the patient that his kidney function was now normal.

### Case Study 22

A 60 year old Hispanic man was being treated for low testosterone blood levels. The patient began twice per day treatment for one month followed by once per day treatment thereafter. Following four months of treatment the patient had normal blood levels of testosterone, even though his supplemental medications had been discontinued.

### Case Study 23

An obese 38 year old Caucasian woman began twice per day treatments for one month and, thereafter, once per day treatments for four months. At the end of the first month, the patient noticed that she was losing weight even though she had not changed her diet. The patient continued to lose weight throughout her treatment period, even though she never altered her diet.

### Case Study 24

A 60 year old Hispanic man was diagnosed as having osteoporosis, with a bone mineral density of 70% of normal for his age and sex. The patient began twice per day treatments for the first month followed by once per day treatments thereafter. After six months of treatment, the bone mineral density was 98% of normal for his age and sex.

### Case Study 25

A 45 year old Caucasian woman had been diagnosed, 7 years previously, as having depression. The patient reported that, within 30 days of receiving twice a day treatments, she felt much less depressed and had a much more positive outlook on life.

### Case Study 26

A 25 yard old Caucasian woman was diagnosed as having panic attacks. The patient began twice per day treatments for one month. At the end of one month, the patient was able to be in crowds and in elevators, without experiencing panic attacks.

### Case Study 27

A 58 year old Caucasian man was diagnosed as having Parkinson's Disease by his Neurologist. The patient began twice per day treatments for one month and, thereafter, once per day treatments for six months. During this time the patient's tremors stopped, his memory markedly improved, and he was able to walk much more normally.

### Case Study 28

A 47 year old Caucasian woman had taken oral medication for hypertension, since she was 17 years old, However, after 4 months of treatment, she was able to stop taking the medication for hypertension and still had normal blood pressure,

### Case Study 29

A 14 year old Caucasian girl was diagnosed with hypoglycemia. However, after 30 days of treatment, the girl stated that she no longer was shaky before meals, nor did she have a headache if she did not eat on schedule. The girl's glucose tolerance test showed that she no longer had hypoglycemia.

### Case Study 30

A 45 year old Caucasian man was diagnosed as having Psoriasis. The patient began twice per day treatments for one month and, thereafter, once per day treatments for three months. At the end of this time, the patient's Psoriatic lesions had virtually disappeared.

### Case Study 31

A 3 year old Caucasian boy was diagnosed as having influenza with early pneumonia, with audible lung involvement. After three days of twice a day treatments, the influenza symptoms resolved and the lungs were clear.

### Case Study 32

A 3 year old Caucasian boy, the twin of the subject of Case Study 32, was diagnosed as having influenza. After two days of twice a day treatments the influenza symptoms resolved.

It would seem unlikely that the same, or substantially the same, treatment would have a beneficial effect on such a large number of diverse ailments. Even though these ailments seem unrelated, there are, in fact, root causes which are ameliorated by the treatments of this invention. By passing impulses from the periphery to the central nervous system, this invention appears to help the body manufacture various neuropeptides and other chemicals, which control the essential basics of the body's health and well being.

Although this invention has been disclosed and described in its preferred forms with a certain degree of particularity, it is understood that the present disclosure of the preferred forms is only by way of example and that numerous changes in the details of operation and in the combination and arrangement of parts may be resorted to without departing from the spirit and scope of the invention as hereinafter claimed.

I claim:

1. A method of treating a human for an ailment selected from the following: bipolar disorder, Brittle Bone Disease, cystic fibrosis, depression, endometriosis, erectile disfunction, fibromyalgia, GERD's, glaucoma, Gulf War Syndrome, hypercholesterolemia, hypertriglyceridemia, hypoglycemia, impotence, influenza, insomnia, irregular heart beat, irritable bowel syndrome, low testosterone levels, obesity, osteoporosis, panic attacks, Parkinson's disease, peripheral vascular disease, psoriasis, sickle cell anemia, sleep apnea, skin ulcerations, TMJ disease, trigeminal neuralgia, Alzheimer's disease, autism, AIDS, ALS, Alzheimer's disease, autism, cancer and congestive heart failure, the method comprising
a) diagnosing one of the ailments in a patient;
b) applying first and second electrodes of a first pair of electrodes to opposite extremities of the patient adjacent termini of different first nerves and applying a third and fourth electrodes of a second pair of electrodes to opposite extremities of the patient adjacent a pair of different termini of the same nerves, the first and third electrodes being applied to the patient adjacent different termini of the same nerve and the second and fourth electrodes being applied to the patient adjacent different termini of the same nerve;
c) delivering electrical interferential pulses through the electrode pairs; and
d) periodically repeating b) and c) until symptoms of the ailment ameliorate.

2. The method of claim 1 wherein the diagnosed one of the ailments is peripheral vascular disease.

3. The method of claim 1 wherein the diagnosed one of the ailments is Brittle Bone Disease.

4. The method of claim 1 wherein the diagnosed one of the ailments is cystic fibrosis.

5. The method of claim 1 wherein the diagnosed one of the ailments is depression.

6. The method of claim 1 wherein the diagnosed one of the ailments is endometriosis.

7. The method of claim 1 wherein the diagnosed one of the ailments is erectile disfunction.

8. The method of claim 1 wherein the diagnosed one of the ailments is fibromyalgia.

9. The method of claim 1 wherein the diagnosed one of the ailments is GERD'S.

10. The method of claim 1 wherein the diagnosed one of the ailments is glaucoma.

11. The method of claim 1 wherein the diagnosed one of the ailments is Gulf War Syndrome.

12. The method of claim 1 wherein the diagnosed one of the ailments is hypercholesterolemia.

13. The method of claim 1 wherein the diagnosed one of the ailments is hypertriglyceridemia.

14. The method of claim 1 wherein the diagnosed one of the ailments is hypoglycemia.

15. The method of claim 1 wherein the diagnosed one of the ailments is impotence.

16. The method of claim 1 wherein the diagnosed one of the ailments is insomnia.

17. The method of claim 1 wherein the diagnosed one of the ailments is irregular heart beat.

18. The method of claim 1 wherein the diagnosed one of the ailments is Irritable Bowel Syndrome.

19. The method of claim 1 wherein the diagnosed one of the ailments is low testosterone.

20. The method of claim 1 wherein the diagnosed one of the ailments is osteoporosis.
